(19) United States
(12) Patent Application Publication
(10) Pub. No.: US 2013/0226055 A1
(43) Pub. Date: Aug. 29, 2013

(54) CERVICAL SPINE IMMOBILIZATION DEVICE

(71) Applicant: William Marsh Rice University, (US)

(72) Inventors: Oviea Akpotaire, Sugar Land, TX (US); Kelsey Horter, Fort Worth, TX (US); Elias Hoban, Austin, TX (US); Georgia Lagoudas, College Station, TX (US); Sailesh Prahbu, Houston, TX (US); Mike Zylberman, Danville, CA (US)

(73) Assignee: William Marsh Rice University, Houston, TX (US)

(21) Appl. No.: 13/774,369

(22) Filed: Feb. 22, 2013

Related U.S. Application Data
(60) Provisional application No. 61/602,751, filed on Feb. 24, 2012.

(51) Int. Cl. A61F 5/055

(52) U.S. Cl. CPC A61F 5/055
USPC 602/18

(57) ABSTRACT

A device is disclosed that can be used to immobilize the neck of patients suffering from blunt trauma injuries to the cervical spine. The device implements biomechanically justified features to immobilize the spine without further harming the patient. In certain embodiments, the device immobilizes the neck by limiting head motion in relation to the upper body.

### CROSS-REFERENCE TO RELATED APPLICATIONS

This application claims the benefit of U.S. Provisional Patent Application No. 61/602,751, entitled “CERVICAL SPINE IMMOBILIZATION DEVICE,” and filed Feb. 24, 2012, which is herein incorporated by reference in its entirety for all purposes.

### BACKGROUND

The present disclosure relates generally to a device that can be used to immobilize the neck of patients suffering from injuries.

In the cases of injuries to the spine, it may be desirable to immobilize the spine without further harming the patient. Existing techniques include the use of a cervical collar (c-collar). However, the c-collar, and the manner in which it is used and/or applied, may not satisfactorily protect the patient. For example, the commonly used semi-rigid cervical collar may stretch the spinal vertebrae and exacerbate the initial spinal injury. In addition, the limited adjustability of the c-collar may result in EMTs using oversized collars on patients. An oversized collar may result in vertebral distraction. In addition, the c-collar may also cause circumferential compression forces around the neck, which can lead to pressure sores and increased intracranial pressure.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

The present device can be used to immobilize the neck of patients suffering from blunt trauma injuries to the cervical spine. The device implements biomechanically justified features to immobilize the spine without further harming the patient.

In certain embodiments, the device immobilizes the neck by limiting head motion in relation to the upper body. Further, in certain embodiments, the device is designed to lay flat when not in use so that it can be wrapped around the patients head in an emergency. To immobilize the injury site, the device may employ rigid chest, back, lateral head supports. In such implementations, these braces will prevent the patient from tilting their head forward or backward and keep the device from slipping down the head. A pair of under arm straps that originate on the back ends of the side supports may also be employed. Such straps, when present, go under the arms to attach to the chest brace, will prevent rotation and side-to-side motion of the head.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts an immobilization device in accordance with aspects of the present disclosure;

FIG. 2 depicts three views of the immobilization device of FIG. 1 when applied to a patient;

FIG. 3 depicts a side view of the immobilization device of FIG. 1 when applied to a patient;

FIG. 4 depicts a side view an immobilization device in accordance with aspects of the present disclosure;

FIG. 5 depicts a front view an immobilization device in accordance with aspects of the present disclosure;

FIG. 6 depicts a top view an immobilization device in accordance with aspects of the present disclosure;

FIG. 7 depicts an isometric view an immobilization device in accordance with aspects of the present disclosure;

FIG. 8 depicts an isometric view an immobilization device applied to a patient in accordance with aspects of the present disclosure;

FIG. 9 depicts a front view an immobilization device applied to a patient in accordance with aspects of the present disclosure;

FIG. 10 depicts a side view an immobilization device applied to a patient in accordance with aspects of the present disclosure; and

FIG. 11 depicts a back view an immobilization device applied to a patient in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As discussed herein, a device is provided that immobilizes the neck by limiting head motion in relation to the upper body. The device may be used to effectively immobilize the neck of a patient without distracting the vertebrae or compressing the airway of the neck. The device may be adjustable to accommodate different body types, and does not interfere with EMT or hospital procedures (e.g. intubation, neck palpation, x-ray, and CT scans). The device may also be sized for pediatric use, i.e., a pediatric sized device may also be used for cervical immobilization of children.

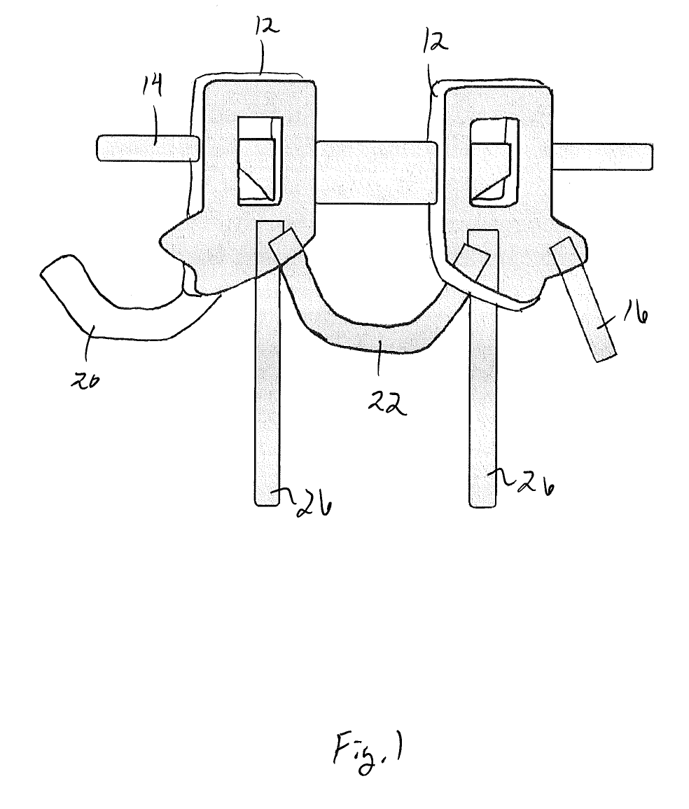

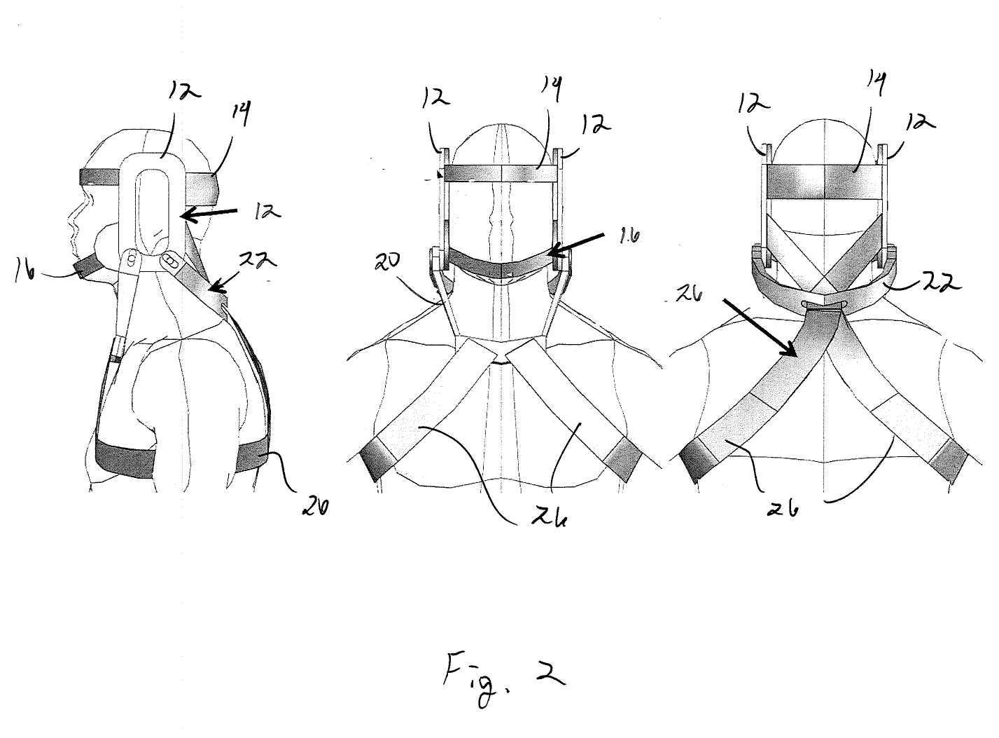

In one embodiment, depicted in FIGS. 1-3, the device 10 is designed to lay flat when not in use so that it can be wrapped around the patients head in an emergency. In the depicted embodiment, the device includes two rigid supports 12 with ear holes intended to stabilize the sides of the patient's head. A head strap 14 runs through the side supports 12 and around the back of the head to be secured at the front of the head. Additionally, a chin strap 16 between the two side supports 12 will allow the head to rest comfortably in the device 10.

In one embodiment, to immobilize the injury site, the device 10 uses a rigid chest support 20 and/or back support 22. These braces, when present, prevent the patient from tilting their head forward or backward and keep the device 10 from slipping down the head. In addition, a pair of under arm straps 26 may be provided that originate on the back ends of the side supports 12 and go under the arms to attach to the chest support 20, to prevent rotation and side-to-side motion of the head. To accommodate differing neck lengths and chest girths, the chest support 20 and back support 22 may be allowed to rotate at their attachment to the side supports 12.

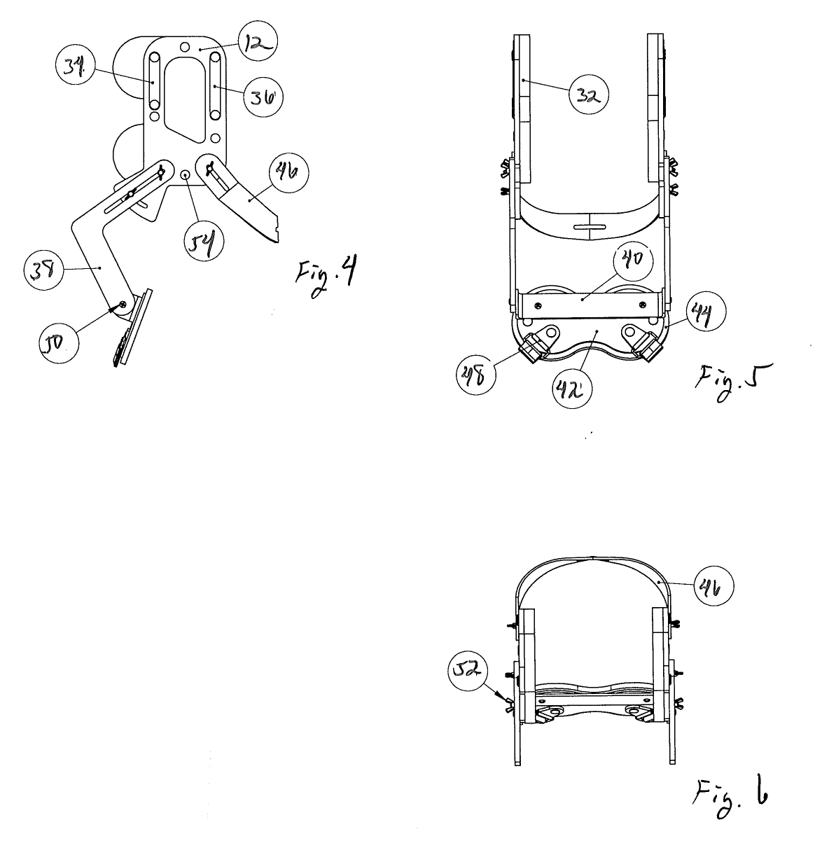

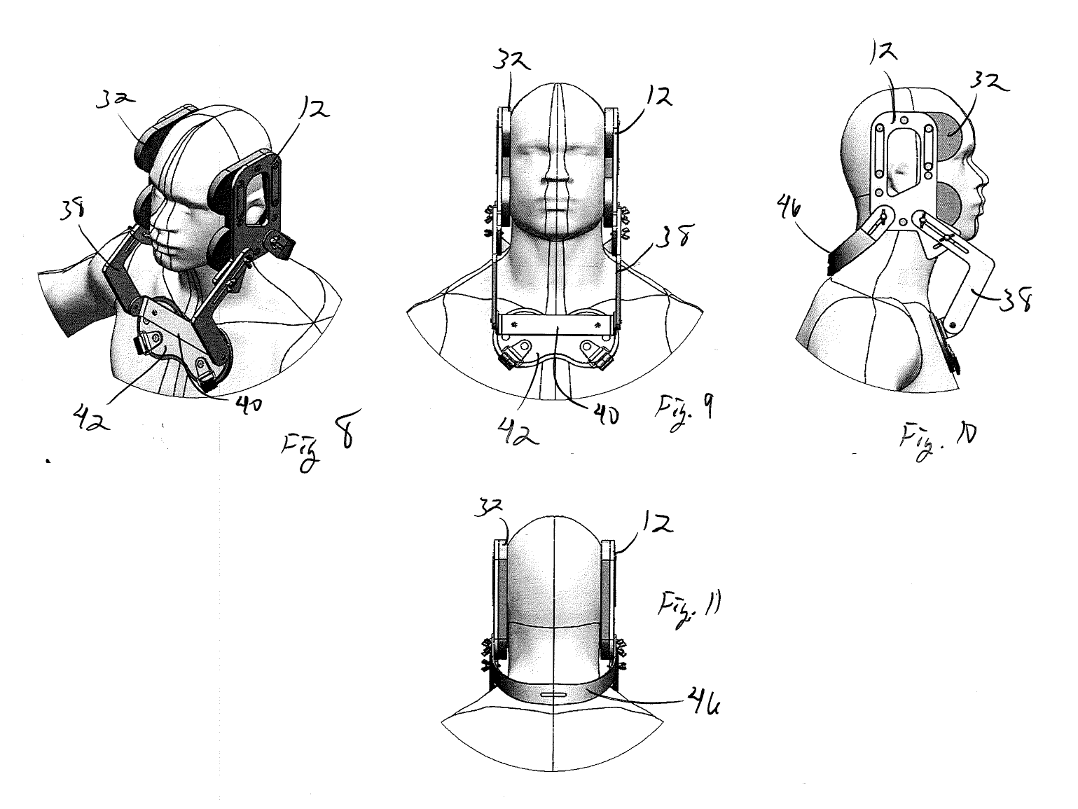

While the preceding provides an overview of aspects of the device 10, the additional FIGS. 4-11 illustrate an example providing additional detail with respect to the brace structures. To simplify illustration and to better view the bracing structures, the various straps are omitted from the illustrations. For example, FIGS. 4-7 depict the head supports 12 and head support foam 32 that covers the patient-facings surfaces of the support 12. As depicted, the head supports 12 each also may include respective slots 34, 36 through which the head strap 14 (not shown) may slide.

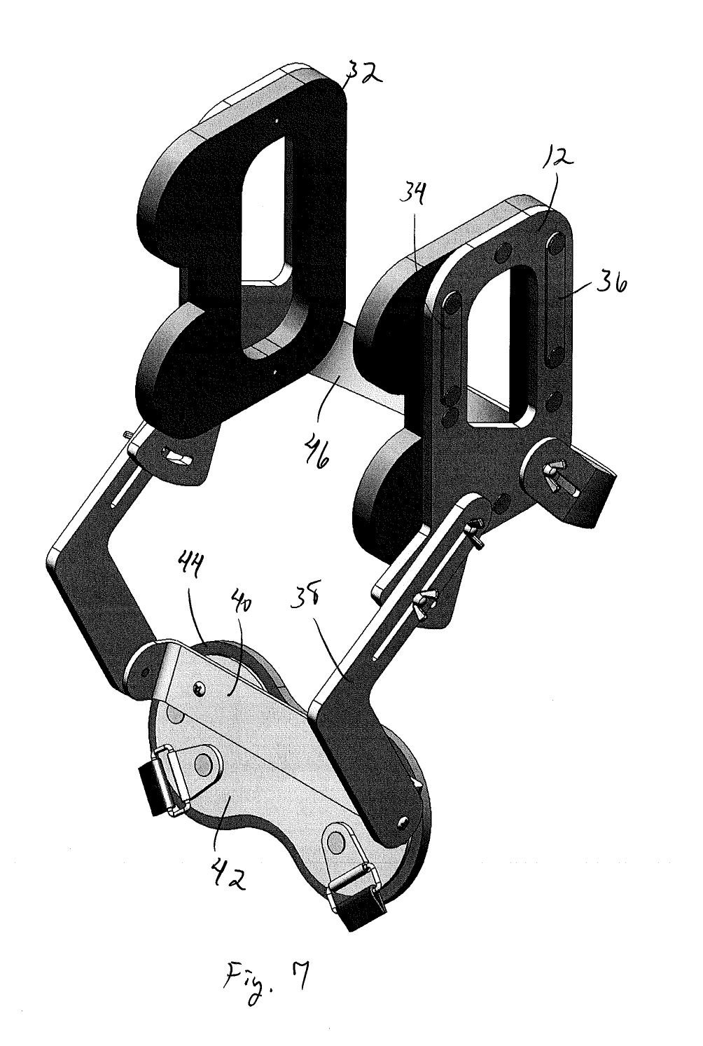

A chest restraint 38 is depicted that is attached and moves relative to the head supports 12. In the depicted example, the chest restraint 38 is attached to a chest brace attachment piece 40 to which the chest brace 42 is attached. In one implementation, chest brace foam 44 covers the patient-facings surfaces of the chest brace 42. Back restraint 46 is also attached to the respective head supports 12. Various attachment structures or devices, such as chafe 48, screws 50, wing screws 52, and/or rivet 54 may also be present in an implementation. FIGS. 8-11 depict an embodiment of the device 10 when applied to a patient.

To manufacture the device 10, rigid supports for the head (support 12), chest (chest support 20), and back (back support 22) may be cut or molded from a stiff plastic, and glued/cemented or riveted to a similarly shaped foam cushion/padding material. The chest support 20 and back support 22 may then be screwed or otherwise assembled into their specified positions on the head supports 12. Head strap 14, chin strap 16, and under-arm straps 26 may then be attached in their appropriate positions.

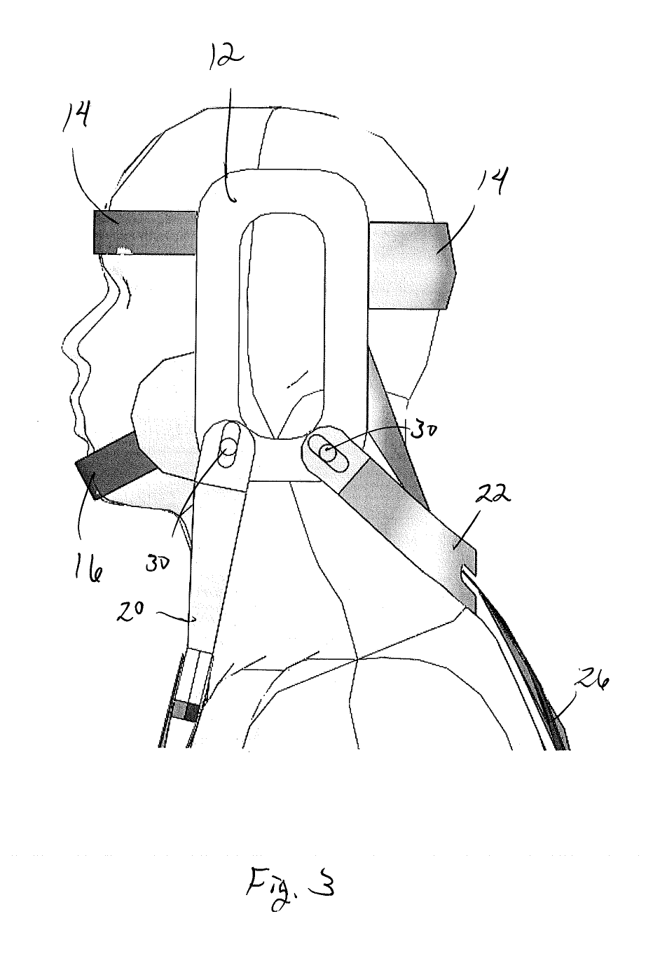

To immobilize the patient, the device 10 will lay flat so that it can be slide under the patients' head and neck while the patient is either lying down or sitting. The device 10 will be then be wrapped around the head so that the head supports 12 comfort the sides of the head without preventing access to the ears. The head support position can be adjusted horizontally along the head strap 14. Once these are fixed, the head strap 14 and chins strap 16 can be tightened and buckled or attached with Velcro. To accommodate differing neck lengths and chest girths, the chest support 20 and back support 22 can rotate at their attachment to the side supports 12 and also be lengthened or shortened as needed. When the supports are in place, they can be secured with slot screws 30 (see FIG. 3). For example, in one embodiment, the supports may be secured using wing nuts fastened onto bolts in the slots. Finally, the underarm straps 26 will be pulled around the body to attach to the chest support plate 20. When applied, the device 10 stabilizes the neck without touching the neck, using the head, chest, and back of the patient for stability.

To minimize the complexity of the device 10, the parts may be color-coded and labeled with pictures that depict the correct locations for attachment and orientation of the different parts (e.g. a depiction of an ear near the ear hole and labeling left and right on the side supports). Patient comfort may be a factor in the selection of the foam, padding material that will be glued to the interior of the hard plastic device.

Variations to the device 10 include the types of plastic and foam cushion materials employed. These materials may selected for material properties (e.g. rigidity), durability, comfort, and cost. Lastly, the attachment of the chest support to the side supports may be done in different manners. For example, in one embodiment, the chest support 20 may be constructed so as to have a half on both side supports 12 that can be connected in the middle. Alternately, the chest support 20 may be formed as one piece where one end is fixed to a side support 12 and the other end will have to be fastened to the other side support 12.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art, including combinations of aspects or features of the embodiments and examples disclosed herein. Such other examples are intended to be within the scope of the claims if they have structural elements that do not differ from the literal language of the claims, or if they include equivalent structural elements with insubstantial differences from the literal languages of the claims. The specific embodiments described above have been shown by way of example, and it should be understood that these embodiments may be susceptible to various modifications and alternative forms, including combinations of various features and aspects of the examples or embodiments discussed herein. It should be further understood that the claims are not intended to be limited to the particular forms disclosed, but rather to cover all modifications, equivalents, and alternatives falling within the spirit and scope of this disclosure.

1. An immobilization device, comprising:
two head support plates, each head support plate configured to be placed on a respect side of a patient's head;
a head strap running between the two head support plates and configured to tighten the two head support plates against the patient's head
a chest support attached to the two head support plates and configured to rotate with respect to the head support plates to contact the patient's chest; and
a back support attached to the two head support plates and configured to contact the patient's back.

2. The immobilization device of claim 1, wherein the immobilization device lays flat when not in use.

3. The immobilization device of claim 1, wherein the two head support plates each include an ear opening.

4. The immobilization device of claim 1, comprising a chin strap running between the two head support plates.

5. The immobilization device of claim 1, wherein the chest support and back support, in combination, prevent the patient from tilting their head forward or backward.

6. The immobilization device of claim 1, comprising a pair of underarm straps configured to attach the two head supports plates to the chest support when secured.

7. The immobilization device of claim 6, wherein the pair of underarm straps, when secured, prevent rotation and side-to-side motion of the patient's head.

8. The immobilization device of claim 1, comprising a respective head support foam piece attached to each head support plate.

9. The immobilization device of claim 1, comprising a respective chest support foam piece attached to the chest support.

10. A method for immobilizing a patient's neck, comprising:
sliding an immobilization device under the head of a patient such that a back support contacts the back of the patient;
securing two head support plates of the immobilization device to the head of the patient using a head strap and a chin strap;
contacting a chest support with the chest of the patient, wherein the chest support is attached to the two head support plates; and
securing two underarm straps underneath the arms of the patient such that the straps are connected to the chest support and the two head support plates.

11. The method of claim 10, wherein contacting the chect support with the chest of the patient comprises rotating the chest support with respect to the two head support plates.

12. An immobilization device, comprising:
a head support structure comprising two head support plates connected by at least a head support strap;
a chest-bracing structure movably connected to the head support structure;
a back restraint connected to the head support structure;
wherein the immobilization device, when applied, stabilizes the neck of a patient without contacting the neck of the patient.

13. The immobilization device of claim 12, comprising respective head support padding provided on the patient-facing surfaces of the head support plates.

14. The immobilization device of claim 12, wherein one or more of the head support plates, the chest-bracing structure, or back restraint are formed from a rigid plastic.

15. The immobilization device of claim 12, comprising respective chest padding provided on the patient-facing surfaces of the chest-bracing structure.

16. The immobilization device of claim 12, wherein the two head support plates include respective slot structures through which the head support strap runs.

17. The immobilization device of claim 12, wherein one or more of the head support structure, the chest-bracing structure, the back restraint, or connecting straps are color coded.

18. The immobilization device of claim 12, wherein one or more of the head support structure, the chest-bracing structure, the back restraint, or connecting straps are labeled with instructive graphics indicating the use of the immobilization device.

19. The immobilization device of claim 12, wherein the two head support plates include ear openings.

20. The immobilization device of claim 12, wherein the chest-bracing structure and/or the back restraint can be adjusted relative to the head support structure to accommodate patient body type.
